(19) United States
(12) Patent Application Publication
(10) Pub. No.: US 2015/0134068 A1
(43) Pub. Date: May 14, 2015

(54) SPACER IMPLANT FOR THE TEMPORARY REPLACEMENT OF A KNEE PROSTHESIS

(71) Applicant: TEKNIMED, Vic En Bigorre (FR)

(72) Inventors: Alain Leonard, Nosy Be (MG); Gautier Halbin, Fontenilles (FR); Cyril Sender, Toulouse (FR); Nouredine Sahraqui, Toulouse (FR)

(21) Appl. No.: 14/395,405

(22) PCT Filed: Apr. 19, 2013
(86) PCT No.: PCT/FR2013/050871

§ 371 Date: Oct. 17, 2014

(30) Foreign Application Priority Data
Apr. 19, 2012 (FR) 1253593

(51) Int. Cl. A61F 2/38

(52) U.S. Cl. CPC A61F 2/3886; A61F 2/3859; A61F 2/389; A61F 2002/3863; A61F 2002/3068
USPC 623/20.27

(57) ABSTRACT

The present invention relates to a spacer implant (I) for the temporary replacement of a knee prosthesis, said implant (I) having a femoral condyle (1) and a tibial plateau (2), the lower surface (7) of the femoral condyle (1) resting on the upper surface (8) of the tibial plateau (2), the lower surface (7) of the femoral condyle (1) having a convex shape while the upper surface (8) of the tibial plateau (2) has a concave shape, a stud (9) being supported by the tibial plateau (2) and penetrating into a receiving means (17) provided on the femoral condyle (1), the femoral condyle (1) comprising two flanges (12, 13) having different sizes and being joined together via a base (7) traversed by a notch (17) forming the receiving means, the shorter flange (13) consisting of two elements (15) referred to as condyles, which are spaced apart from one another by the extension of the notch (17), and a stabilizing bar (16) joining the two condyles (15) together at the free end of said flange (13).

The present invention relates to the field of orthopedic surgery, particularly knee joint surgery. More specifically, the invention relates to a spacer implant used for the temporary replacement of a defective knee prosthesis.

When a knee prosthesis becomes infected, it is advisable to remove the infected prosthesis and treat the infection. Such treatment consists of replacing the infected prosthesis in two stages, the first stage involving removal of the infected knee prosthesis and taking out the infected areas.

In the second stage, a spacer, such as a block of cement having the shape of a knee prosthesis, is then installed for a period until another prosthesis can safely be implanted.

Such a spacer maintains limb length, joint pressures, and muscular strength to prevent muscle atrophy while awaiting reimplantation of a new prosthesis.

Such spacers also exist for the treatment of prostheses such as hip or shoulder prostheses. In a known manner, these spacers are often made manually by the surgeon performing the removal of the infected prosthesis, using a biocompatible material such as a PMMA bone cement. The surgeon then tries to shape the cement as closely as possible to the shape of the removed prosthesis.

As mentioned above, such a spacer is implanted temporarily in order to replace the removed prosthesis, for the period required to clear the knee infection. This intermediate treatment time can last up to 6 months. Such a spacer can also contain an antibiotic delivered gradually into the knee. Once this period has elapsed, a new “permanent” knee prosthesis can be implanted.

Yet other spacers are made manually in situ by the surgeon, but are created in molds providing simple forms.

The main drawback of such spacers is that they do not provide mobility and function respecting the anatomy of the joint.

To avoid this disadvantage, molds have been used providing forms similar to those of the prostheses. The surgeon can pour his PMMA bone cement into them to produce more functional spacers.

To allow the surgeon to have an implant ready for use, these PMMA bone cement spacers preexist, supplied in sterile form and indicated as being ready for use.

These spacers meet the functional and anatomical requirements of the joint. Furthermore, some preformed spacers are made from bone cement impregnated with an antibiotic. It is known that this type of cement can release the antibiotic compound it contains once it is in place in the knee.

The problem underlying the present invention is to design a spacer implant for the temporary replacement of a knee prosthesis, which is easy to implant while ensuring complete stability of the joint during motion. The knee must be able to perform flexion/extension movements in the sagittal plane, with a limited degree of rotation to avoid placing too much stress on the joint during this sensitive period involving the temporary replacement of a knee prosthesis following an infection, in particular to avoid interfering with the healing process.

The invention therefore relates to a spacer implant for the temporary replacement of a knee prosthesis, said implant having a femoral condyle and a tibial plateau, the lower surface of the femoral condyle resting on the upper surface of the tibial plateau, characterized in that the lower surface of the femoral condyle has a convex shape while the upper surface of the tibial plateau has a concave shape, a stud being supported by the tibial plateau and penetrating into receiving means provided on the femoral condyle, the femoral condyle comprising two flanges having different sizes and curving away from the tibial plateau, the two flanges being joined together via a base whose lower portion forms the lower surface of the femoral condyle, the base being traversed by a notch extending in the longitudinal direction of the femoral condyle, said notch forming the receiving means provided on the femoral condyle, the shorter flange being composed of two elements referred to as condyles, spaced apart from one another by the extension of the notch, a stabilizing bar joining the two condyles at the free end of said flange.

Advantageously, the outer face of the longer flange comprises a median longitudinal channel, referred to as the hinge channel.

Advantageously, the femoral condyle and the tibial plateau respectively support a pin and a post, the pin and the post being intended for insertion into the bone respectively facing the condyle or plateau.

Advantageously, the pin of the femoral condyle is located between the longer flange and the notch traversing the base of said condyle.

Advantageously, the pin and the post may be of different dimensions, according to the patient's anatomy.

Advantageously, at least the femoral condyle or the tibial plateau comprises one or more internal chambers provided with at least one opening for the controlled release over time of a drug substance.

Advantageously, the material or materials of the femoral condyle or tibial plateau are porous in order to absorb and then release a drug substance.

Advantageously, an adjustment pad is arranged under the bottom of the plateau.

Advantageously, at least the femoral condyle or the tibial plateau comprises metal reinforcements.

The invention will now be further described in a non-limiting manner with reference to the accompanying figures, in which:

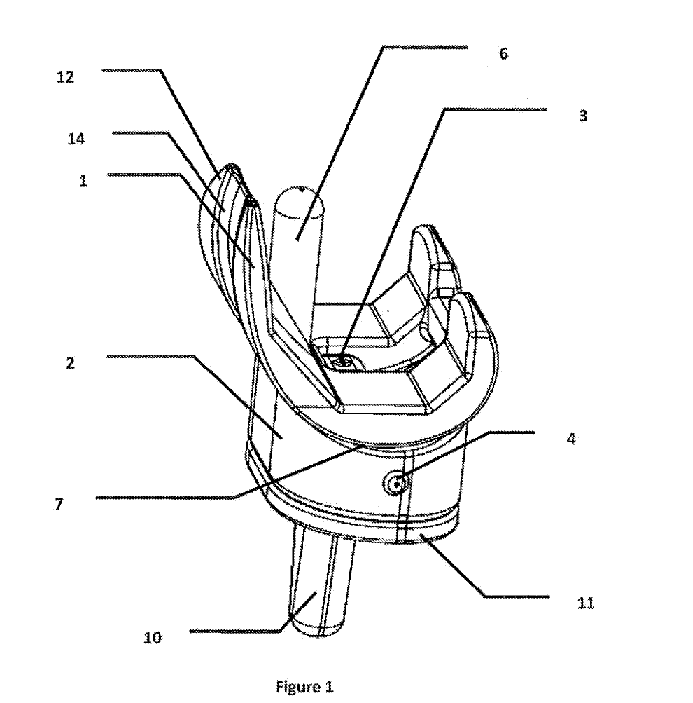

FIG. 1 is a schematic representation of a top perspective view of a spacer implant according to the invention,

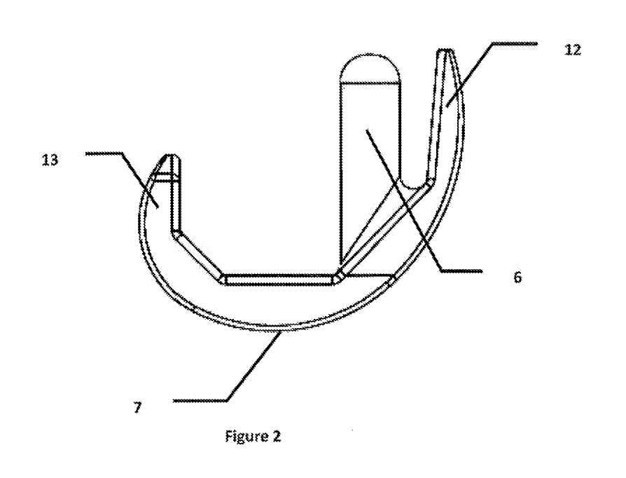

FIG. 2 is a schematic representation of a longitudinal side view of a femoral condyle that is part of a spacer implant according to the invention,

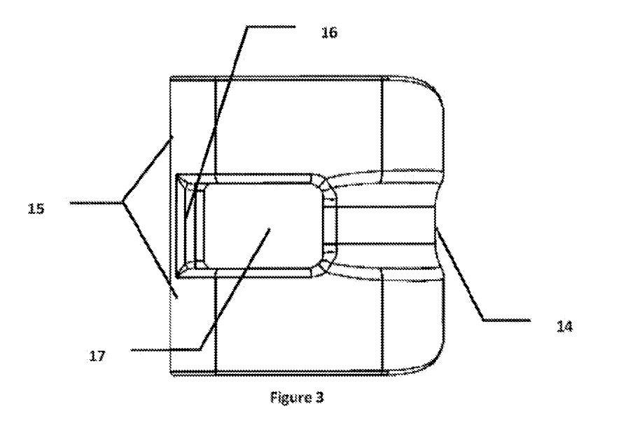

FIG. 3 is a schematic representation of a bottom view of a femoral condyle that is part of a spacer implant according to the invention,

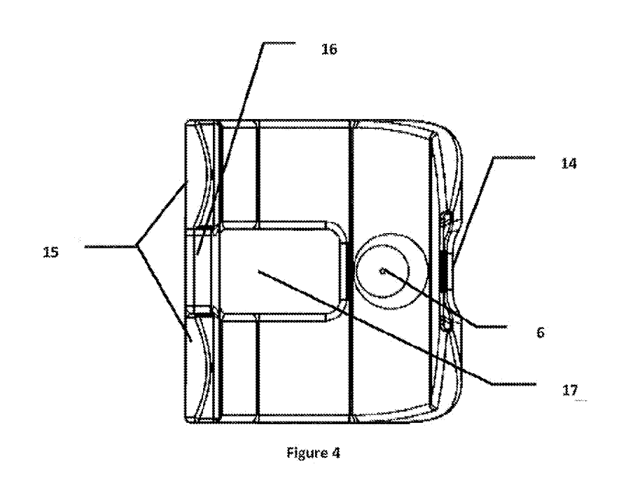

FIG. 4 is a schematic representation of a top view of a femoral condyle that is part of a spacer implant according to the invention,

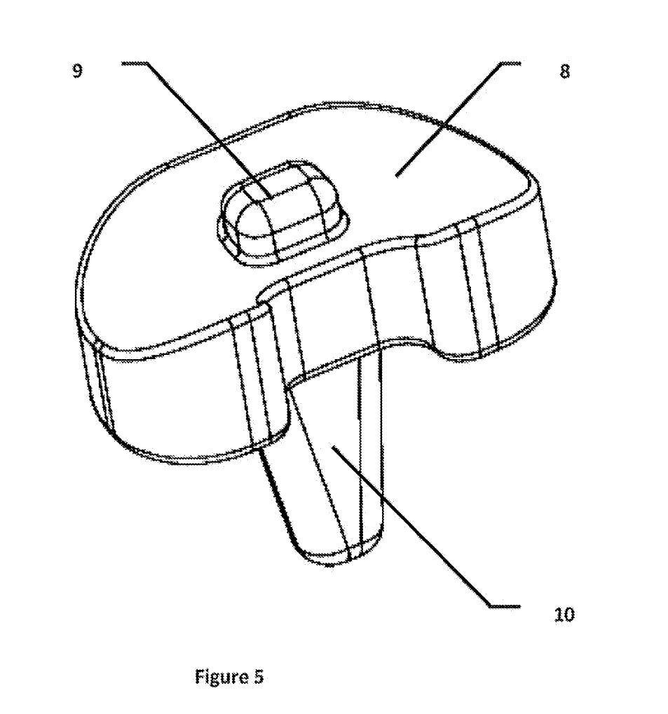

FIG. 5 is a schematic representation of a perspective view of a tibial plateau that is part of a spacer implant according to the invention,

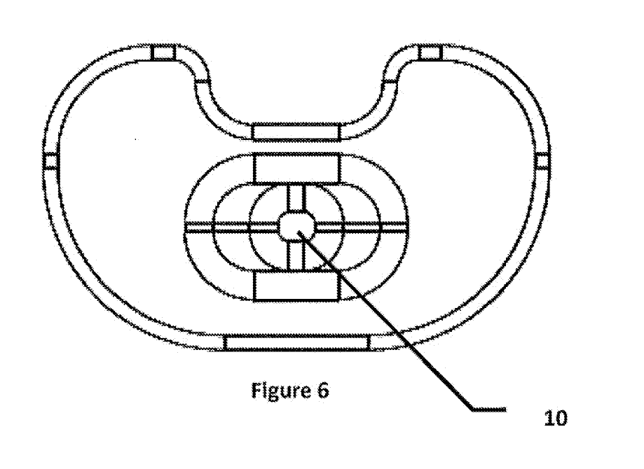

FIG. 6 is a schematic representation of a bottom view of a tibial plateau that is part of a spacer implant according to the invention,

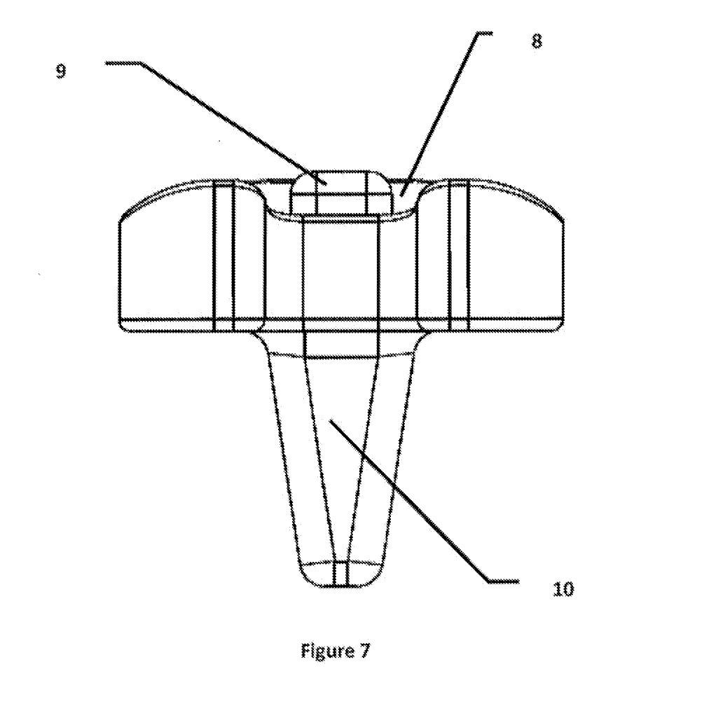

FIG. 7 is a schematic representation of a rear view of a tibial plateau that is part of a spacer implant according to the invention,

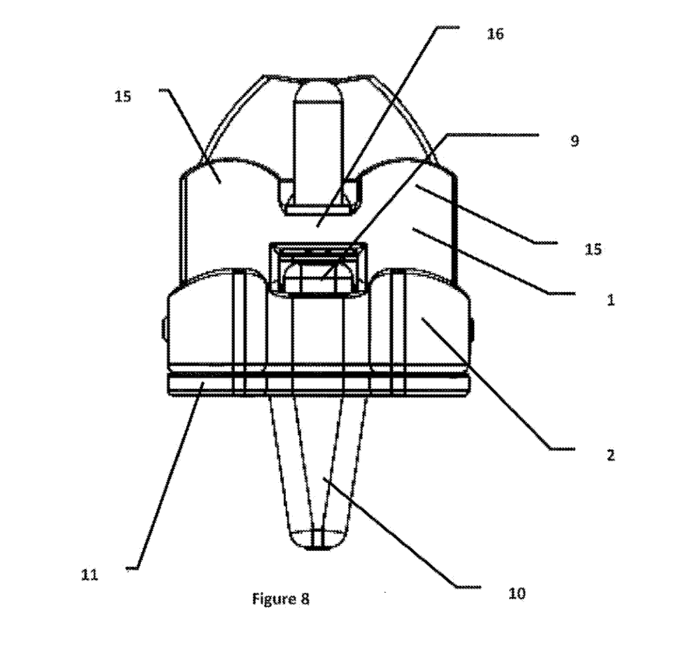

FIG. 8 is a schematic representation of a rear view of a spacer implant according to the invention,

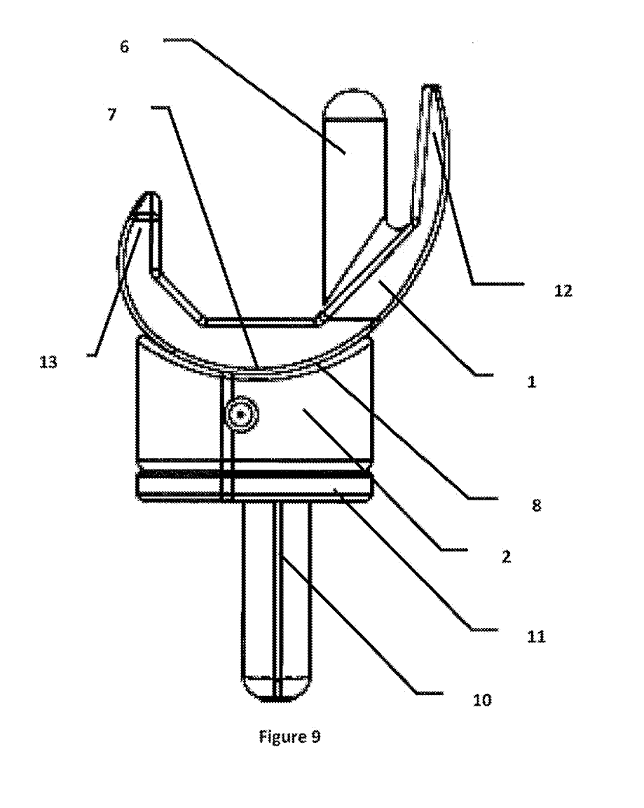

FIG. 9 is a schematic representation of a longitudinal side view of a spacer implant according to the invention,

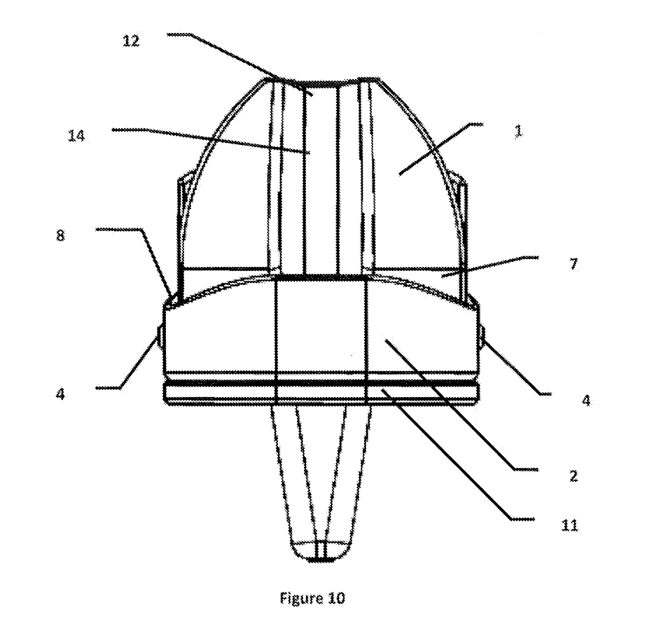

FIG. 10 is a schematic representation of a front view of a spacer implant according to the invention.

FIG. 1 shows a spacer implant I for the temporary replacement of a knee prosthesis in accordance with the invention. This spacer implant I is composed of two parts, a femoral condyle 1 and a tibial plateau 2, which are implanted for the period required to heal the infection and allow a healing phase.

The tibial plateau 2 has a main body forming a plateau which is extended underneath by a post 10. This post 10, which extends downward when the spacer implant I is in the mounted position, is intended to be secured to the upper end of the patient's tibia according to known means.

The tibial plateau 2 also comprises an adjustment pad 11 on its bottom. This pad 11, which serves to adjust the height of the tibial plateau 1, can be used in certain anatomical cases. The pad 11 has a shape similar to the bottom of the tibial plateau 2 and presses against it.

The pad 11 thus adds additional thickness to the tibial plateau 2 when it is not thick enough for the patient. This adjustment pad 11 may be of PMMA cement and may be impregnated with antibiotic such as gentamicin for example. Advantageously, the adjustment pad 11 can also be reinforced with biocompatible metal reinforcement.

The main body of the tibial plateau 2 is provided with an internal chamber with at least one opening 4. This internal chamber can contain a solution of drug substance(s), for example based on antibiotics. This substance is released gradually over time during the temporary placement of the spacer implant I in order to treat the knee infection.

This also applies to the femoral condyle 1 which may also comprise a chamber 3. A chamber 3 may also be provided on the tibial plateau 1 and enter the notch 17 of the femoral condyle 1.

Treatment of the knee infection conventionally occurs by administration of an antibiotic and the presence of such chambers 3 helps to administer this treatment. These chambers can be molded into the implant I or may be independent capsules that can be placed on the implant I in openings provided for this purpose.

The chambers 3 may be filled in situ by the surgeon, with an antibiotic, an anesthetic, or an anti-infective agent. These additives may be in liquid, solid, or gaseous form. The surgeon may inject the antibiotic of his choice or a mixture of antibiotics or even a different antibiotic in each chamber.

These chambers 3 may be positioned at different locations. The chambers 3 may be incorporated into the tibial post 10 and/or a femoral pin 6 which will be described later. It is possible to incorporate them into other locations of the femoral condyle 1, tibial plateau 2, or into an adjustment pad 11.

Multiple volumes may exist for a chamber 3, for example ranging from 0.1 ml to 5 ml, so that it is possible for the antibiotic to be released in a distributed manner over a period ranging from 1 to 6 weeks. These chambers 3 may be made with different types of materials, resorbable or non-resorbable, for example PMMA or equivalent material.

Currently existing implants are made of PMMA cement impregnated with antibiotic for local treatment of the infection in addition to prophylactic treatment.

Additionally or alternatively to the at least one chamber 3, the spacer implant I of the invention may comprise portions having a different porosity, such portions allowing better distribution of the drug substance, advantageously one or more antibiotics.

Thus, one or more basic materials of the spacer implant I may be impregnated with antibiotic for local treatment of the infection. The material or materials used may also be composed of opacifying agents.

The femoral condyle 1 will now be described more specifically, with reference to FIGS. 1 to 4.

The femoral condyle 1 has an upper surface intended to be turned towards the patient's femur, and is substantially concave in shape.

The femoral condyle 1 is formed of two lateral flanges 12 and 13 that curve upwards, therefore away from the tibial plateau 2, and are joined together by a base 7. One of the flanges 12, 13 is longer than the other flange 13. The longer flange 12 is called the hinge surface.

The longer flange 12 has a longitudinal channel 14 on its external surface, called the hinge channel This channel 14 guarantees the stability of the patella and provides housing for the patella.

Between the longer flange 12 and the base 7, on the upper surface of the femoral condyle 1, a pin 6 is provided that projects upward when the implant I is in its inserted position in the knee. This pin 6 penetrates the lower end of the patient's femur to maintain the implant I in place in cooperation with the post 10 of the tibial plateau 2 penetrating the patient's tibia, so that the implant I effectively fulfills its function as the knee joint of the patient and is anchored at two points.

With particular reference to FIG. 1, the femoral condyle 1 and the tibial plateau 2 respectively have a pin 6 and a post 10 for stabilizing the implant spacer I, the pin 6 and the post 10 being embedded in the lumen of the bone.

Advantageously, the post 10 of the tibial plateau 2 is trapezoidal and has an oblong cross-section while the pin 6 of the femoral condyle 1 is substantially cylindrical.

The functions of the pin 6 and post 10 are to fill the void left by the defective knee prosthesis and by the removal of the infected area, and also to stabilize the spacer implant I by stabilizing an element of said implant I, respectively the femoral condyle 1 or the tibial plateau 2.

The second side flange which is the shorter flange 13 than the flange 12 referred to as the hinge surface, supports two elements referred to as condyles 15 interconnected by a stabilizing bar 16 extending in the transverse direction of the femoral condyle 1. This stabilizing bar 16 which joins together the two condyles 15 at the free end of the flange 13, cooperates with the stud 9 on the tibial plateau 2 so that it prevents any lateral play between the two condyles. It can also act as a stop for the stud 9. limiting the degree of rotation of the joint during flexion movements in a sagittal plane. With this stabilizing bar 16, the knee is “blocked” after a certain degree of rotation which limits the movement of the joint, providing increased stability and encouraging healing and resorption of the infection.

Advantageously, a notch 17 is provided that passes through, from top to bottom, a portion of the second side surface 13 and the base 7 and extending from the stabilizing bar 16 to a chamber 3 located substantially at the bottom of the pin 6 for anchoring with the base 7. This notch 17 forms the receiving means provided by the femoral condyle as will be seen below.

Advantageously, the pin 6 of the femoral condyle is located between the longer flange 12 and the notch 17 traversing the base 7 of said condyle 1.

Similarly to the chamber of the tibial plateau 2, the chamber 3 of the femoral condyle 1, provided with an opening, may contain a antibiotics-based solution which is released gradually over time during the temporary placement of the implant spacer I, in order to treat the knee infection.

The lower portion of the base 7 of the femoral condyle 1 abuts against the upper portion of the tibial plateau 2 when the implant I is in the mounted position. The lower portion of the base 7 has a substantially convex shape for better contact with the upper portion of the tibial plateau 2, which is concave.

FIGS. 5 to 7 show a view of the tibial plateau 2, illustrating the means for positioning the femoral condyle 1 on said tibial plateau 2.

The upper surface 8 of the tibial plateau 2 has a concave surface which allows receiving the lower surface of the femoral condyle.

As indicated above, the upper surface 8 of the tibial plateau 2 has a stud 9 referred to as the posterior-stabilizing stud. This stud penetrates the lower portion of the base of the femoral condyle. In case of knee flexion within a sagittal plane, the condyles 15 move rotationally as they slide over the upper surface 8 of the tibial plateau 2. This rotation is blocked by the stud 9 abutting against the stabilizer bar 16 connecting the two condyles 15 at the free end of the flange 13, encouraging joint stability and healing after the infected implant removal operation.

In FIG. 8 one can see that the posterior-stabilizing stud 9 of the tibial plateau 2 enters the receiving means provided in the femoral condyle 1, these means consisting of the notch 17.

In addition, in FIG. 9 one can see that the convex lower surface of the base 7 of the femoral condyle 1 rests on the concave upper surface 8 of the tibial plateau 2. The concave shape of the upper surface 8 of the tibial plateau 2 and the convex shape of the lower surface of the base 7 of the femoral condyle 1 guarantee better stability of the implant during its motion.

By allowing the femoral condyle 1 to remain properly placed relative to the tibial plateau 2, the posterior-stabilizing stud 9 provides the advantage of preventing dislocation of the knee joint. Furthermore, it can simulate the function of the cruciate ligaments.

The femoral condyle provides an accommodating cradle 1, formed by the notch 17 which receives the posterior-stabilizing stud during assembly of the tibial plateau member 2 with the femoral condyle member 1. An alternative could consist of a recess other than the notch 17.

In FIG. 10 one can see that the chamber inside the tibial plateau 2 has two openings 4 arranged on opposite sides of said plateau 2.

Such a spacer implant I can come in several sizes to provide a surgeon with wide flexibility in adapting to a patient's anatomy. The adaptability of this size range also provides better patient comfort.

This also applies to the size of the post 10 of the tibial plateau 2 and/or of the pin 6 of the femoral condyle 1, these elements being particularly visible in FIG. 1.

Advantageously, the post 10 and/or the pin 6 can be detached from their supporting member and replaced by other posts 10 or pins 6 of different dimensions, which provides satisfactory flexibility and better stability for the implant I.

Advantageously, still with reference to FIG. 1 in particular, the femoral condyle 1 and the tibial plateau 2 as well as the adjustment pad 11 are of reinforced materials, for example containing a frame with metal reinforcement to strengthen the implant I.

The spacer implant I can be made of biocompatible material with sufficient mechanical properties to allow the patient some autonomy in walking about.

Indeed, it has been found that many knee spacers have insufficient mechanical strength and fail after implantation. Such failures are likely to release debris that can damage the joint area, such as the lateral ligaments. This can be avoided with the reinforcements as described above.

The main advantage of this invention is to provide a shape that is functional and anatomical by incorporating the general characteristics of the knee, both technical or dimensional, which allows good knee movement while maintaining certain limits such as during flexion in a sagittal plane, so as not to interfere with healing. Another advantage lies in the ease of installation and removal of the implant.

With such an implant, the comfort and controlled mobility of the patient are improved, due to the quality of the functional surfaces, thereby limiting friction between the femoral condyle and tibial plateau of the implant, and due to the anatomical adaptation of the implant to the patient. In addition, such a spacer implant has good mechanical strength properties.

1. Spacer implant (I) for the temporary replacement of a knee prosthesis, said implant (I) having a femoral condyle (1) and a tibial plateau (2), the lower surface (7) of the femoral condyle (1) resting on the upper surface (8) of the tibial plateau (2), characterized in that the lower surface (7) of the femoral condyle (1) has a convex shape while the upper surface (8) of the tibial plateau (2) has a concave shape, a stud (9) being supported by the tibial plateau (2) and penetrating into receiving means (17) provided on the femoral condyle (1), the femoral condyle (1) comprising two flanges (12, 13) having different sizes and curving away from the tibial plateau (2), the two flanges (12, 13) being joined together via a base (7) whose lower portion forms the lower surface of the femoral condyle (1), the base (7) being traversed by a notch (17) extending in the longitudinal direction of the femoral condyle (1), said notch (17) forming the receiving means provided on the femoral condyle (1), the shorter flange (13) being composed of two elements (15) referred to as condyles, spaced apart from one another by the extension of the notch (17), a stabilizing bar (16) joining the two condyles (15) at the free end of said flange (13).

2. Implant (I) of claim 1, wherein the outer face of the longer flange (12) comprises a median longitudinal channel (14), referred to as a hinge channel.

3. Implant (I) of claim 1, wherein the femoral condyle (1) and the tibial plateau (2) respectively support a pin (6) and a post (10), the pin (6) and the post (10) being intended for insertion into the bone respectively facing the condyle or plateau.

4. Implant (I) according to claim 3, wherein the pin (6) of the femoral condyle (1) is located between the longer flange (12) and the notch (17) traversing the base (7) of said condyle (1).

5. Implant (I) according to claim 3, wherein the pin (6) and the post (10) are of different dimensions, according to the patient's anatomy.

6. Implant (I) according to claim 1, wherein at least the femoral condyle (1) or the tibial plateau (2) comprises one or more internal chambers (3) provided with at least one opening (4) for the controlled release over time of a drug substance.

7. Implant (I) according to claim 1, wherein the material or materials of the femoral condyle (1) or tibial plateau (2) are porous in order to absorb then release a drug substance.

8. Implant (I) according to claim 1, wherein an adjustment pad (11) is arranged under the bottom of the plateau (2).

9. Implant (I) according to claim 1, wherein at least the femoral condyle (1) or the tibial plateau (2) comprises metal reinforcements.

10. Implant (I) of claim 2, wherein the femoral condyle (1) and the tibial plateau (2) respectively support a pin (6) and a post (10), the pin (6) and the post (10) being intended for insertion into the bone respectively facing the condyle or plateau.

11. Implant (I) according to claim 4, wherein the pin (6) and the post (10) are of different dimensions, according to the patient's anatomy.

12. Implant (I) according to claim 2, wherein at least the femoral condyle (1) or the tibial plateau (2) comprises one or more internal chambers (3) provided with at least one opening (4) for the controlled release over time of a drug substance.

13. Implant (I) according to claim 3, wherein at least the femoral condyle (1) or the tibial plateau (2) comprises one or more internal chambers (3) provided with at least one opening (4) for the controlled release over time of a drug substance.

14. Implant (I) according to claim 4, wherein at least the femoral condyle (1) or the tibial plateau (2) comprises one or more internal chambers (3) provided with at least one opening (4) for the controlled release over time of a drug substance.

15. Implant (I) according to claim 5, wherein at least the femoral condyle (1) or the tibial plateau (2) comprises one or more internal chambers (3) provided with at least one opening (4) for the controlled release over time of a drug substance.

16. Implant (I) according to claim 2, wherein the material or materials of the femoral condyle (1) or tibial plateau (2) are porous in order to absorb then release a drug substance.

17. Implant (I) according to claim 3, wherein the material or materials of the femoral condyle (1) or tibial plateau (2) are porous in order to absorb then release a drug substance.

18. Implant (I) according to claim 4, wherein the material or materials of the femoral condyle (1) or tibial plateau (2) are porous in order to absorb then release a drug substance.

19. Implant (I) according to claim 5, wherein the material or materials of the femoral condyle (1) or tibial plateau (2) are porous in order to absorb then release a drug substance.

20. Implant (I) according to claim 6, wherein the material or materials of the femoral condyle (1) or tibial plateau (2) are porous in order to absorb then release a drug substance.
